(19) United States
(12) Patent Application Publication
(10) Pub. No.: US 2023/0228654 A1
(43) Pub. Date: Jul. 20, 2023

(54) Blood Sampling Device and Method for PEth Measurement

(71) Applicant: CAPITAINER AB, Solna (SE)

(72) Inventor: Olof BECK, Saltsjö-Boo (SE)

(21) Appl. No.: 18/002,537

(22) PCT Filed: Jul. 2, 2021
(86) PCT No.: PCT/EP2021/068386

§ 371 Date: Dec. 20, 2022

(30) Foreign Application Priority Data
Jul. 2, 2020 (SE) 2050827-1

(51) Int. Cl. G01N 1/34; B01L 3/00

(52) U.S. Cl. CPC G01N 1/34; B01L 3/50273; B01L 2300/0816; B01L 2300/126; B01L 2400/0406; B01L 2200/16

(57) ABSTRACT

A device configured to collect a blood sample comprising a capillary means, wherein the capillary means is configured to collect and dry the blood sample and comprises an effective amount of a distributed inhibitor of phospholipase D. The device may be configured to receive, transport and collect a blood sample comprising a compartment in fluid connection with the capillary means, wherein the capillary means is configured to collect and dry the blood sample and comprises an effective amount of a distributed PLD inhibitor. The device may be a microfluidic device comprising an inlet portion, an outlet portion comprising a capillary means configured to collect and dry the blood sample, and optionally a metering function, wherein the microfluidic device comprises an effective amount of a distributed PLD inhibitor. The PLD inhibitor is distributed in a water soluble film, preferably a PVA film, or in an absorbent paper or polymer or in the capillary means. The PLD inhibitor may be selected from a salt of a transition metal belonging to column 5 or 6 of the periodic table, a salt of vanadium and a salt of tungsten, a salt comprising a vanadium oxyanion and a salt comprising a tungsten oxyanion, and/or at least one of NaVO3 (sodium metavanadate) and Na2WO4 (sodium tungstate). A method of preparing a sample for analysis of phosphatidylethanol (PEth) comprises providing a blood sample with a volume of less than 10 ml to the device; contacting the blood sample with at least one inhibitor of the enzyme phospholipase D selected from at least one of a salt of vanadium and a salt of tungsten; and admitting inhibition of phospholipase D so formation of PEth is blocked.

### TECHNICAL FIELD

The present invention relates to improvements in sampling of blood to determine the alcohol biomarker phosphatidylethanol (PEth).

### BACKGROUND ART

Phosphatidylethanol is a substance that forms from cell membrane phospholipids during exposure to ethanol (i.e. alcohol drinking). Phospholipase D (PLD) is the enzyme responsible for the reaction and is caused by the feature of the enzyme to prefer ethanol as substrate over water when acting to form phosphatidic (PA) acid from phosphatidylcholines (PCs) as part of a regulatory system. When formed, PEth will be incorporated into the cell membrane and becomes accumulated over time. Repeated alcohol drinking will lead to elevated blood levels of PEth and it has been suggested as biomarker to detect riskful alcohol consumption from risk concluding limits. For this purpose, several assays have been suggested in for example U.S. Pat. No. 5,066,583; WO 2009/054784; U.S. Pat. No. 8,795,980 and US 2019/0293668.

Sampled blood is an active biofluid that retains many biological activities after sampling. One such activity is the enzyme Phospholipase D (PLD), which is involved in regulation of the lipid content of the cell membrane. PLD has the unique feature to have activity also after cooling and freezing. Accordingly, PEth can be formed in vitro post sampling if ethanol is present in the blood at the time of sampling, which is not uncommon in real practice.

A Schröck et al in Alcohol, 2018, Vol. 73, pages 1-7 discloses the development of standardized tests for measuring individual formation of PEth following alcohol consumption. The authors were aware of the problem with post-sampling formation of PEth and introduced the well-known PLD inhibitors halopemide and FIPI (5-fluoro-2-indolyl-deschlorohalopemide) to improve the quality of the PEth analysis in order to prevent in vitro formation of PEth in alcohol positive blood samples after blood sampling. However, halopemide and FIPI were considered unsuitable for clinical use due to low potency and high costs. For this reason, the problem with post-sampling PLD needs to be solved before feasible routine assays of PEth can be established.

In addition, it is desirable to conform routine PEth assay to the economic and rapid screening technology admitted by DBS (Dried Blood Spot Sampling) where blood samples are conveniently collected and dried, as represented by the device disclosed by US2015/0037903. Although, it was estimated that dried blood spots may solve the problems with post-sampling formation of PEth, it was found that PLD remains active and that PEth forms also during the drying process and possibly also in the dried blood. There is an obvious need to find a solution to the possibility of post-sampling formation of PEth as it may lead to erroneous measurements and false interpretations.

The present invention therefore meets need of finding better strategies to develop a reliable, economic and convenient assay of PEth.

### SUMMARY OF INVENTION

An object of the present invention is thus to provide a simple solution to a reliable, economical and convenient assay of PEth that avoids the problems with post-sampling formation of PEth.

In one aspect of the invention, the first embodiment, a device configured for collection and subsequent testing of a blood sample of less than 10 ml includes at least one inhibitor of the enzyme phospholipase D selected from at least one of a salt of vanadium and a salt of tungsten.

The inhibitor of the enzyme phospholipase D may be selected from at least one of a salt comprising a vanadium oxyanion and a salt comprising a tungsten oxyanion. Further, the inhibitor may be selected from at least one of NaVO₃ (sodium metavanadate) and Na₂WO₄ (sodium tungstate).

The at least one salt of inhibitor of the enzyme phospholipase D may have a mass of less than 10 mg.

The at least one salt is present in amount that admits concentration of less than 1 mM in the blood sample.

The device may be a test tube; the test tube may comprise a bottom, a lid and a coating.

The device may have an inner coating of less than 100 micrometers comprising the at least one inhibitor of the enzyme phospholipase D.

The inner coating may be sprayed on the on the bottom and/or on the lid of the test tube.

In another aspect of the invention, a method is disclosed for preparing a sample for analysis of phosphatidylethanol (PEth) is provided comprising providing a blood sample for a patient with a volume of less than 10 ml; contacting the blood sample with at least one inhibitor of the enzyme phospholipase D selected from at least one of a salt of vanadium and a salt of tungsten; and admitting inhibition of phospholipase D so formation of PEth is blocked.

In a further aspect of the invention, a method of applying a coating of at least one inhibitor of the enzyme phospholipase D to a test tube is disclosed. The method comprises the steps of stabilizing the test in a substantially vertical position; inserting a spray nozzle inside the test tube, the spray nozzle being in fluid connection to a container holding a solution comprising at least one inhibitor of the enzyme phospholipase D; spraying the solution comprising at least one inhibitor of the enzyme phospholipase D inside the test tube; and allowing the solution comprising at least one inhibitor of the enzyme phospholipase D to dry.

In a further aspect of the invention, a test tube with a coating comprising at least one inhibitor of the enzyme phospholipase D obtained by the above-mentioned method is also disclosed.

In another aspect of the invention, the second embodiment, a device is configured to collect a blood sample comprises a capillary means, wherein the capillary means is configured to collect and dry the blood sample and comprises an effective amount of a distributed inhibitor of phospholipase D.

In yet another aspect of the embodiment, a device is configured to receive, transport and collect a blood sample comprises a compartment in fluid connection with the capillary means, wherein the capillary means is configured to collect and dry the blood sample and comprises an effective amount of a distributed inhibitor of phospholipase D.

The capillary means may be a porous paper or polymer configured to admit capillary transport to and from the compartment of the blood sample.

In yet another aspect of the embodiment, a device configured to receive a blood sample is a microfluidic device comprising an inlet portion, an outlet portion comprising a capillary means configured to collect and dry the blood sample, and optionally a metering function, wherein the microfluidic device comprises an effective amount of a distributed PLD inhibitor.

The PLD inhibitor is distributed in a water soluble film, preferably a polyvinyl alcohol (PVA) film, or in an absorbent paper or polymer or in the capillary means.

The PVA film, the absorbent paper or the polymer with the distributed PLD inhibitor is located in the inlet portion, or in the outlet portion.

The PVA film, the absorbent paper or the polymer with distributed PLD inhibitor is located in the outlet portion.

The metering function comprises a metering channel for metering a controlled volume of 5-50 microliters of blood transported to the PVA film, the absorbent paper, or the polymer with distributed PLD inhibitor located in the outlet.

With respect to all aspects of the embodiment, the inhibitor of phospholipase D may be selected from a salt of a transition metal belonging to column 5 or 6 of the periodic table.

The inhibitor of phospholipase D may be selected from at least one of a salt of vanadium and a salt of tungsten.

Advantageously, the inhibitor may be selected from at least one of a salt comprising a vanadium oxyanion and a salt comprising a tungsten oxyanion.

The inhibitor may further be selected from at least one of NaVO₃ (sodium metavanadate) and Na₂WO₄ (sodium tungstate).

In another aspect, a method is disclosed for preparing a sample for analysis of phosphatidylethanol (PEth) is disclosed. The method comprises the steps of applying a blood sample with a volume of less than 10 ml to the device; contacting the blood sample with at least one inhibitor of the enzyme phospholipase D selected from at least one of a salt of vanadium and a salt of tungsten; and admitting inhibition of phospholipase D so formation of PEth is blocked.

### BRIEF DESCRIPTION OF DRAWINGS

The invention is now described, by way of example, with reference to the accompanying drawings, in which:

FIG. 1 shows a test tube device according to a first embodiment.

FIG. 2 shows a device according to a second embodiment. The device is shown (a) in a cross section along the capillary channel and (b) in a cross section across the capillary channel, defined by the plane A-A in (a).

### DESCRIPTION OF EMBODIMENTS

In the following, a detailed description of embodiments of the invention is disclosed.

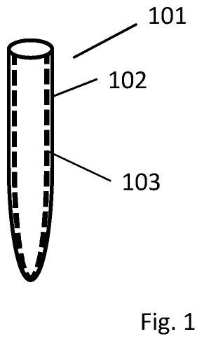

FIG. 1 shows a device 101 according to a first alternative, comprising a blood collection test tube 102 that contains an PLD inhibitor enzyme. The blood collection test tube can be a conventional blood test tube made of glass or suitable polymers, such as Vacutainer®, specially labelled for PEth analysis. Similar to the blood collection test tubes with added components such as EDTA, heparin, etc. that are already known in the art, the test tube comprises a PLD inhibitor, as an added component.

The PLD inhibitor enzyme may be present as a solid at the bottom of the test tube. The PLD inhibitor enzyme may be a solid at the bottom of the test tube. Alternatively, the PLD inhibitor enzyme may be added as a solution to the test tube. In yet another alternative, the PLD inhibitor enzyme may be present in a gel attached to the walls of the test tube. Other means of immobilizing the PLD inhibitor enzyme are also contemplated.

It has been found that the salts of a transition metal belonging to column 5 or 6 of the periodic table, in particular, the salts of vanadium and tungsten are effective inhibitors of the enzyme phospholipase D. Other agents that may also be used as inhibitors of the enzyme D may include certain anti-cancer drugs, such as 5-fluoro-2-indolyldes-chlorohalopemide (FIPI), or agents with certain ionic properties, such as phosphate analogues.

In further embodiments, the walls of the test tube may have been impregnated with a PLD inhibitor, or the walls of the test tube may have a coating comprising the PLD inhibitor. The PLD inhibiting agents can be included in thin film coatings of the container (see for example U.S. Pat. No. 6,428,527) or added by other conventional ways to supply blood test tubes with additives.

In addition to the PLD inhibitor, the test tube may further include other conventional additives such as anticoagulants.

The test tube is used for blood collection. Approximately 5-10 ml of blood is added to the test tube. The test tube is then sealed/closed with a test tube cap and the now sealed test tub is gently rocked for approximately 5 minutes. Thereafter, the sealed test tube containing the blood sample is stored at either room temperature or +4° C. pending further analysis.

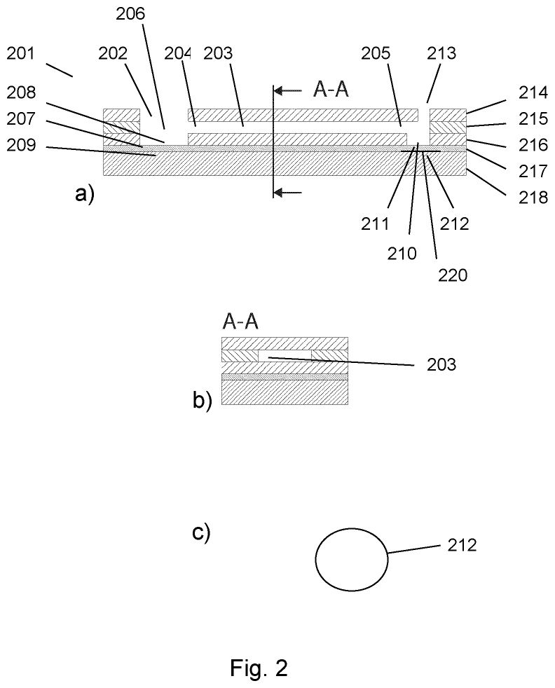

FIG. 2 shows a device 201 according to a second alternative, comprising a capillary means comprising a capillary means configured to collect and dry the blood sample and an effective amount of a homogeneously distributed inhibitor of phospholipase D. The capillary means may be an absorbent paper or polymer.

In one embodiment device 201 comprises a capillary channel 203 having a defined volume and having an inlet portion 204 and an outlet portion 205. The inlet portion is connected to an inlet port 202 for liquid, such as a bodily fluid. The inlet port is arranged in connection to an inlet chamber 206 for receiving an undefined volume of liquid, such as about 30 μl.

The inlet chamber is in fluid connection to a first dissolvable valve 207 comprising a dissolvable membrane 208 and a capillary means 209 in the form of a layer of absorbing paper, such as Whatman 903 DBS paper. The membrane has a first side facing the liquid in the inlet chamber and a second side facing the capillary means such that when the membrane is dissolved by the liquid, liquid is transported through the valve to the second side of the membrane by capillary action. The membrane may be a layer of PVA obtained in the form of a sheet or film or prepared by spin-coating of a liquid solution of polyvinylalcohol (PVA), which is a water dissolvable thermoplastic polymer. It has excellent film forming and adhesion properties. It is nontoxic and used in various medical applications. The material has a high tensile strength and is flexible. PVA is a liquid soluble polymer and a 30 μm thick layer is dissolved by a drop of water within approximately 90 seconds. Thus, the layer of PVA is preferably less than 20 μm, more preferably less than 10 μm, or even less than 5 μm to dissolve in less than 60 seconds, less than 30 seconds or less than 15 seconds. Preferably a PVA film thickness of 1-10 μm is used. The membrane 207 thus has a thickness much smaller than a lateral dimension of the membrane and thus allows for efficient dissolution by the liquid without loading the liquid with unnecessary amounts of dissolved material.

The outlet portion 205 of the capillary channel 203 is in capillary connection with a second dissolvable valve 210 comprising a dissolvable membrane 211 and a capillary means 212 in the form of a layer of absorbing paper, such as Whatman 903 DBS paper. The capillary means in the form of an absorbing paper is impregnated or coated with a PLD inhibitor. The outlet portion 205 of the capillary channel 203 further connected to a vent port 213 for venting air from the channel during capillary filling with the liquid.

The microfluidic device 201 shown in FIG. 2 is in the form of a multilayered device comprising three layers 214, 215 and 216 defining the microfluidic structures forming the inlet chamber 206, the capillary channel 203 and the vent port 213. The dissolvable membranes 208 and 211 of the respective dissolvable valves 207 and 210 are formed by a layer 217 of dissolvable PVA, and the capillary means 209 and 212 by a layer 218 of absorbing paper.

To use the device, a vanadium or tungsten salt is added to the absorbent paper. For example, the absorbent paper or polymer is soaked in a vanadium or tungsten salt solution comprising, for example, 1 mM of the respective salt. The absorbent paper or polymer is allowed to dry. Blood is then applied on the absorbent paper or polymer. The blood is then allowed to dry on the absorbent paper or polymer to create a dried blood spot. The absorbent paper or polymer is then punched out to take out a predetermined area of the dried blood spot. The dried blood spot is then used for extracting PEth and analyzing the resulting extract.

Alternatively, the absorbent paper or polymer is sprayed with a water-based vanadium or tungsten salt solution. A polyvinyl alcohol (PVA) film is then placed on top of the absorbent paper or polymer. In one preferred embodiment the PVA film has a thickness of approximately 10 microns.

In yet another alternative, the PVA film comprises vanadium or tungsten salts.

A disc is then punched out of the absorbent paper or polymer covered by the PVA film; the area of the disc should contain 15-25 micrograms of the vanadium or tungsten salt.

The disc comprising the PLD inhibitor may be used in the inlet chamber comprising capillary means 209.

In a preferred embodiment the disc comprising the PLD inhibitor is used in the capillary means 212 of the outlet portion 205 of the capillary channel 203 such that the entire volume of blood contacts the PLD inhibitor for improved performance.

### Example 1

Blood Samples

The blood specimens used for the present investigation were deidentified surplus volumes of venous whole blood selected among those sent to the Department of Clinical Pharmacology, Karolinska University Laboratory (Stockholm) for routine analysis of PEth as an alcohol biomarker.

Additional blank specimens were collected from non-drinking healthy volunteers at the laboratory. The blood was collected in EDTA tubes and stored at 4° C. where PEth is reported to be stable for at least 3 weeks. Ethanol was not tested for. The procedures followed were approved by the ethics committee at the Karolinska University Hospital (No. 2013/341-31/4).

Measurement of PEth in Fresh Venous Blood

Routine analysis of PEth 16:0/18:1 in whole blood specimens was done essentially as previously described. 100 μL whole blood was mixed with 50 μL IS solution (PEth-d31; Avanti Polar Lipids, Alabaster, Ala., USA), 75 μL acetonitrile, and 150 μL acetone. The mixture was gently shaken (40 rpm) for 20 min at room temperature and then centrifuged at 4000 g for 20 min. The supernatant was transferred to a new vial and centrifuged again for another 10 min. LC-MS/MS quantification of PEth 16:0/18:1 was done by comparison with a calibration curve covering 0-14.2 μmol/L prepared similarly in blank (i.e. PEth negative) blood specimens spiked with known amounts of PEth 16:0/18:1 (Avanti Polar Lipids) [14]. Two quality control samples (low and high PEth level) were prepared in the same way. The detection limit (LOD) and lower quantification limit (LLOQ) of the method were 0.01 μmol/L and 0.03 μmol/L, respectively.

Measurement of PEth in DBS Collected on Standard Filter Paper

For method evaluation and validation, DBS samples were prepared by pipetting 45 μL of the venous blood specimen onto the filter paper (Whatman 903 Protein Saver Card; GE Healthcare Ltd., Cardiff, UK), which was then allowed to dry at room temperature for at least 3 hours in horizontal position with air on both sides. For the analysis, 3 filter paper punches (4.7 mm diameter) were taken from each DBS and placed in a test tube and added with 10 μL IS solution and 125 μL methanol. The tube was covered and gently shaken (40 rpm) for 1 hour, followed by centrifugation at 4000 g for 10 min. The liquid phase was transferred to an autosampler vial and centrifuged for another 10 min, prior to analysis. LC-MS/MS quantification of PEth 16:0/18/1 in the sample was done by comparison with a DBS calibration curve prepared as described for venous blood.

Measurement of PEth in DBS Samples Collected Using a Volumetric Device

The value of volumetric DBS measurement of PEth was examined using a disposable prototype DBS device (provided by Capitainer AB, Stockholm, Sweden) [25] constructed with an inlet cavity for applying 30-50 μL (i.e. about one drop) of blood. After the application of blood, a capillary channel is automatically filled with 14 μL of the sample which is eventually emptied onto a Whatman 903 filter paper disc (6.0 mm diameter). The paper disc is impregnated with, for example, 1 mg of salt. For this study, 50 μL venous whole blood was applied to the inlet cavity using a pipette.

After drying at room temperature for at least 2 hours, the volumetric filter disc was transferred to a glass test tube and extracted with 200 μL isopropanol containing IS (0.035 μmol/L PEth-d5; Chiron AS, Trondheim, Norway). The test tube was gently shaken (40 rpm) for 60 min at room temperature and 150 μL of the liquid phase transferred to an autosampler vial. A 2-μL aliquot was injected on the column and the PEth 16:0/18:1 concentration was measured by LC-MS/MS as described above. The standards were prepared by fortifying DBS spots from blank blood with reference PEth 16:0/18:1 in isopropanol. The method was calibrated between 0.025 and 5.00 μmol/L PEth 16:0/18:1. Uncertainty in quantification was documented both for intra- and interassay imprecision and accuracy, using authentic blood quality controls with assigned PEth concentrations.

Results:

TABLE I
Experiment with PEth formation during drying on DBS filter paper

| Blank blood + 2 | PEth response | PEth response |
|---|---|---|
| per mille ethanol | native | with salt |
| Sample 1 | 33 | Not detected |
| Sample 1 | 29 | Not detected |
| Sample 2 | 33 | Not detected |
| Sample 2 | 39 | Not detected |
| Sample 3 | 30 | Not detected |
| Sample 3 | 24 | Not detected |

The results demonstrate that with the inhibitor present during drying additional artificial formation of PEth does not take place.

Thus, the use of the rock salts NaVO₃ and NA₂WO₄ is simple, easy to work with and safe. They withstand the temperature during production.

1.-13. (canceled)

14. A device configured to receive a blood sample comprising a capillary means, wherein the capillary means is configured to collect and dry the blood sample and comprises an effective amount of a distributed inhibitor of phospholipase D (PLD).

15. A device according to claim 14, wherein the device is configured to transport and collect a blood sample, and the device comprises a compartment in fluid connection with the capillary means.

16. The device according to claim 14, wherein the capillary means is a porous paper or polymer configured to admit capillary transport to and from the compartment of the blood sample.

17. The device according to claim 14, wherein the distributed PLD inhibitor is selected from at least one of salt of a transition metal belonging to column 5 or 6 of the periodic table.

18. The device according to claim 17, wherein the distributed PLD inhibitor is selected from at least one of a salt of vanadium and a salt of tungsten.

19. The device according to claim 18, wherein the inhibitor is selected from at least one of a salt comprising a vanadium oxyanion and a salt comprising a tungsten oxyanion.

20. The device according to claim 19, wherein the inhibitor is selected from at least one of NaVO₃ (sodium metavanadate) and Na₂WO₄ (sodium tungstate).

21. A device configured to receive a blood sample, wherein the device is a microfluidic device comprising an inlet portion, an outlet portion comprising a capillary means configured to collect and dry the blood sample, and optionally a metering function, wherein the microfluidic device comprises an effective amount of a distributed inhibitor of phospholipase D (PLD).

22. The device according to claim 21, wherein the PLD inhibitor is distributed in a water soluble film, preferably a polyvinyl alcohol (PVA) film, or in an absorbent paper or polymer or in the capillary means.

23. The device according to claim 22, wherein the PVA film, the absorbent paper or the polymer with the distributed PLD inhibitor is located in the inlet portion, or in the outlet portion.

24. The device according to claim 23, wherein the PVA film, the absorbent paper or the polymer with distributed PLD inhibitor is located in the outlet portion.

25. The device according to claim 24, wherein the metering function comprises a metering channel for metering a controlled volume of 5-50 microliters of blood transported to the PVA film, the absorbent paper, or the polymer with distributed PLD inhibitor located in the outlet.

26. The device according to claim 21, wherein the distributed PLD inhibitor is selected from at least one of salt of a transition metal belonging to column 5 or 6 of the periodic table.

27. The device according to claim 26, wherein the distributed PLD inhibitor is selected from at least one of a salt of vanadium and a salt of tungsten.

28. The device according to claim 27, wherein the inhibitor is selected from at least one of a salt comprising a vanadium oxyanion and a salt comprising a tungsten oxyanion.

29. The device according to claim 28, wherein the inhibitor is selected from at least one of NaVO₃ (sodium metavanadate) and Na₂WO₄ (sodium tungstate).

30. A method of preparing a sample for analysis of phosphatidylethanol (PEth) comprising:
applying a blood sample with a volume of less than 10 ml to a device configured to receive a blood sample comprising a capillary means, wherein the capillary means is configured to collect and dry the blood sample and comprises an effective amount of a distributed inhibitor of phospholipase D (PLD);
contacting the blood sample with at least one PLD inhibitor; and
inhibiting the PLD in the blood sample with the at least one PLD inhibitor so formation of PEth is blocked.

31. A method of preparing a sample for analysis of phosphatidylethanol (PEth) comprising:
applying a blood sample with a volume of less than 10 ml to a device configured to receive a blood sample, wherein the device is a microfluidic device comprising an inlet portion, an outlet portion comprising a capillary means configured to collect and dry the blood sample, and optionally a metering function, wherein the microfluidic device comprises an effective amount of a distributed inhibitor of phospholipase D (PLD);
contacting the blood sample with at least one PLD inhibitor; and
inhibiting the PLD in the blood sample with the at least one PLD inhibitor so formation of PEth is blocked.
